(19) United States
(12) Patent Application Publication
(10) Pub. No.: US 2004/0193200 A1
(43) Pub. Date: Sep. 30, 2004

(54) Scissors, in particular, for surgical purposes

(75) Inventors: Manfred Dworschak, Duerbheim (DE); Theodor Lutze, Balgheim (DE); Pedro Morales, Tuttlingen-Nendingen (DE); Dieter Weisshaupt, Immendingen (DE)

Correspondence Address: LAW OFFICE OF BARRY R LIPSITZ, 755 MAIN STREET, MONROE, CT, 06468, US

(73) Assignee: AESCULAP AG & Co. KG, Tuttlingen (DE)

(21) Appl. No.: 10/763,941

(22) Filed: Jan. 22, 2004

Related U.S. Application Data
(63) Continuation of application No. PCT/EP02/08389, filed on Jul. 27, 2002.

(30) Foreign Application Priority Data
Aug. 4, 2001 (DE) 101 38 356.8

(51) Int. Cl. A61B 17/32

(52) U.S. Cl. 606/174

(57) ABSTRACT

In scissors, in particular, for surgical purposes, comprising two branches pivotable relative to one another, each of which has a cutting section with a cutting edge at the front end thereof and a handle section at the rear end thereof, in order to simplify manufacture thereof, it is proposed that at least one of the branches be joined in its cutting section on its inner surface facing the other branch to a metal foil lying flat thereon and extending at least at the cutting edge end of the inner surface as far as this cutting edge end or projecting slightly over this, with the edge of the metal foil arranged at this end of the inner surface forming the cutting edge of this inner surface.

The present disclosure relates to the subject matter disclosed in international application No. PCT/EP02/08389 of Jul. 27, 2002, which is incorporated herein by reference in its entirety and for all purposes.

### BACKGROUND OF THE INVENTION

The invention relates to scissors, in particular, for surgical purposes, comprising two branches which are pivotable relative to each other and each have a cutting section with a cutting edge at the front end thereof and a handle section at the rear end thereof.

Such scissors are used widely, for example, in the surgical field for cutting tissue, drapes, suture threads, etc., and, in all cases, it is necessary to have a sharp and effective cutting edge at one's disposal. It is therefore standard practice to grind these scissors. This is a complex operation, and whether or not the scissors will be usable for the intended purpose depends on the success of this operation.

The object of the invention is to so design generic scissors that these are particularly easy to manufacture, in particular, without the necessity for a grinding operation for the cutting edge.

### SUMMARY OF THE INVENTION

This object is accomplished with scissors of the kind described at the outset, in accordance with the invention, in that at least one of the branches is joined in its cutting section on its inner surface facing the other branch to a metal foil lying flat thereon and extending at least at the cutting edge end of the inner surface as far as this cutting edge end or projecting slightly over this, and the edge of the metal foil arranged at this end of the inner surface forms the cutting edge of this inner surface. Surprisingly, it has been found that by using such a metal foil covering the inner surface of at least one branch, but, preferably, of course, of both branches, in the cutting section, a cutting edge which has the necessary sharpness can be formed without any special grinding of the cutting edge being required therefor. In this case, it suffices to join the metal foil flat to the inner surface so that the edge of the metal foil itself then forms a sharp cutting edge suitable for all intended purposes without any special grinding operations.

The metal foil used for this purpose is thin in relation to the thickness of the branch, with the thickness of the metal foil preferably being between 0.05 and 0.4 mm.

The metal foil may be made from various metals, and use of a metal foil made of spring steel is particularly advantageous.

It is also expedient for the metal foil to consist of a metal with a high degree of hardness, for example, a hardness of at least HRC60.

The metal foil may be produced in different ways. For example, the metal foil may be a punched part, a laser-cut part or a wire-eroded part. In all cases, the metal foil is worked out of a larger extended metal foil and is then usable immediately after this working-out, for example, by punching, laser-cutting or wire eroding, without there being any necessity for subsequent machining.

The metal foil is preferably adhesively bonded to the inner surface of the branch.

For example, a thermally polymerizable adhesive or a hot-melt adhesive may be arranged between metal foil and inner surface.

It is particularly advantageous for a double-sided adhesive surface, for example, a double-sided adhesive tape, as used for fixing brake discs in the automobile industry, to be arranged between metal foil and inner surface. It is also possible to join the metal foil in a different way to the inner surface of the branch. For example, the metal foil can be welded or soldered to the branch. If plastic material is used as material for the branch, the metal foil may also be injected into the plastic material. It is advantageous for the foil surface to be structured on the side facing the inner surface, so that a particularly intimate bonding is achieved between the material of the branch and the metal foil in this area.

In particular, the metal foil may carry projections on its side facing the inner surface of the branch. The projections dip into the material of the branch and preferably have undercuts for this purpose.

The structure of the projections may, for example, be cauliflower-like, with the projections being distributed irregularly over the metal foil and forming spherical or approximately spherical projections which are joined by stem-like connections to the metal foil.

Such projections may, for example, be formed by an adhesive layer electrolytically applied to the foil.

In a particularly preferred embodiment the projections consist of nickel.

It is also possible to structure the surface of the foil on the side facing the inner surface, in particular, to produce projections on this side by the metal foil being made to undergo an etching treatment.

It is particularly advantageous for the metal foil and the inner surface of the branch to be joined together by hot-stamping. With such hot-stamping, the metal foil and the branch are pressed against one another under pressure and possibly under additional heating. The projections thereby penetrate the plastic material of the branch and this plastic material can flow around the projections, thereby producing an interlocked connection between metal foil and branch after the cooling.

The cutting edge of the metal foil may be smooth. However, in accordance with a preferred embodiment, provision is made for the edge forming the cutting edge of the metal foil to have projections and recesses adjacent one another, in particular, tooth-shaped projections and recesses. The effect thereby achieved is similar to that achieved with a wave-shaped cutting edge, and slipping of the part held for cutting between the cutting edges is thereby prevented.

Furthermore, in a preferred embodiment provision may be made for the inner surface to carry projections which are raised in the direction towards the opposite branch and engage in cutouts in the metal foil and thereby position this on the inner surface. On the one hand, this positioning aids the manufacture, as it is then very easy to bring the metal foil into the position relative to the inner surface in which it is to be joined to the inner surface, and, on the other hand, the metal foil is thereby also stabilized relative to the inner surface, so that the connection between metal foil and inner surface, for example, the adhesive bonding is subjected to less stress when shearing forces act on the metal foil during the cutting.

A projection may, for example, be arranged at the edge of the inner surface opposite the cutting edge, and it is advantageous for the projection to extend parallel to the edge of the inner surface over part of the length of this edge.

It is also possible for a projection to be arranged at the handle section end of the inner surface, and it is then advantageous for this projection to converge in the direction towards the front end of the inner surface, and, in particular, to be of wedge-shaped design. Such a projection carries out a centering function, so that the metal foil placed on the inner surface is pushed directly and exactly into the desired position during the placing.

It is advantageous for the inner surfaces to be of concave shape and for the cutting sections of the two branches to be resiliently pressed against one another. It is thus ensured that the cutting edges always rest against one another at the actual cutting point and thereby enable a perfect cut.

In addition, provision may be made for the cutting sections to have a setting. With such a setting, the cutting sections are tilted slightly in opposite directions about their longitudinal axis so that they include a small angle with one another. Thus, the inner surfaces do not lie with surface-to-surface contact against one another but produce a kind of clearance angle, as can be achieved with conventional scissors by a specific grinding of the cutting edges.

In principle, the described scissors construction may be used with branches made of different materials, i.e., also with metal scissors. However, such a construction is particularly advantageous in scissors with branches consisting of a plastic material. In this way, scissors can be produced using as branch material plastic materials which themselves are unable to form suitable cutting edges, but by placing the described metal foils on the inner surfaces, it is possible to produce scissors which consist almost entirely of plastic material and yet have excellent cutting characteristics.

In particular, in the surgical field polyamides (PA, PPA), polyetheretherketone (PEEK) or liquid crystal polymers (LCP) may, for example, be used as plastic materials. It is expedient for the plastic material to be reinforced with fibers, for example, with glass fibers or with carbon fibers.

The inner surfaces of the branches with the metal foil may extend in the direction towards their handle sections beyond the pivot connection of the branches. In particular, the pivot connection may be arranged in the middle part of the inner surfaces provided with the metal foil, so that the pivot connection can press the two inner surfaces resiliently against one another.

Various pivot connections are feasible. Pivot connections produced by the two branches being joined with a simple positive fit, so that no additional shaft elements need be used, are particularly advantageous. It is, however, readily possible to use the described construction in scissors with branches which are pivotably connected to one another by shafts, screws, etc.

The following description of preferred embodiments of the invention serves in conjunction with the drawings to explain the invention in greater detail.

### BRIEF DESCRIPTION OF THE DRAWINGS

FIG. 1 shows a plan view of scissors with a lug-type joint;

FIG. 2 shows a side view of these scissors in the direction of arrow A in FIG. 1;

FIG. 3 shows a sectional view taken along line 3-3 in FIG. 2;

FIG. 4 shows an enlarged section corresponding to area B in FIG. 3 with a smooth cutting edge;

FIG. 5 shows a view similar to FIG. 4 with a sawtooth-shaped cutting edge;

FIG. 6 shows a sectional view taken along line 6-6 in FIG. 1;

FIG. 7 shows a plan view of a structured surface of a metal foil; and

FIG. 8 shows a sectional view taken along line 8-8 in FIG. 7.

### DETAILED DESCRIPTION OF THE INVENTION

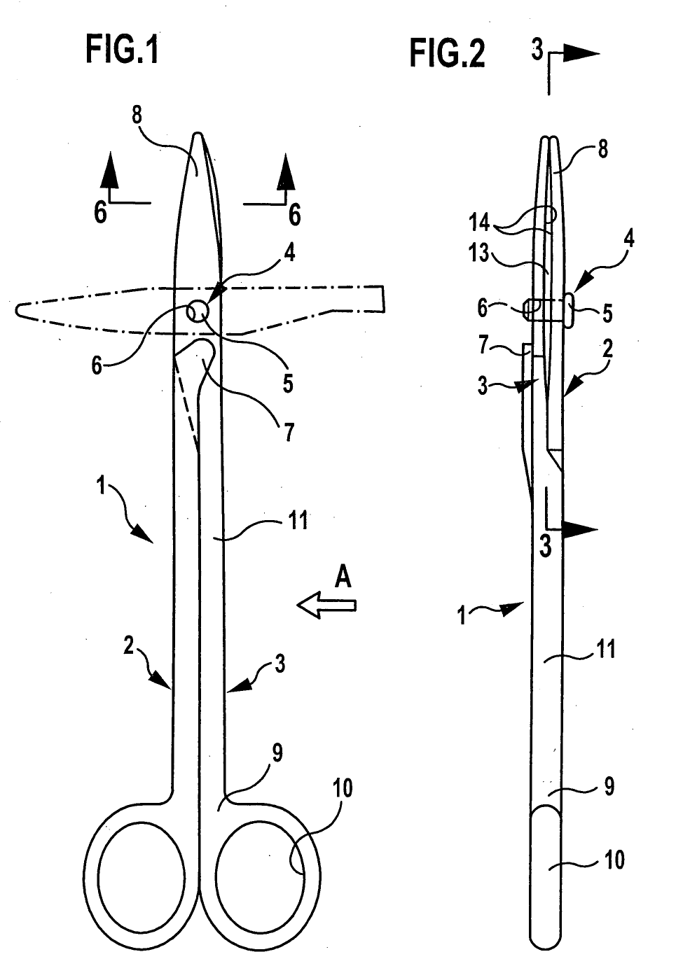

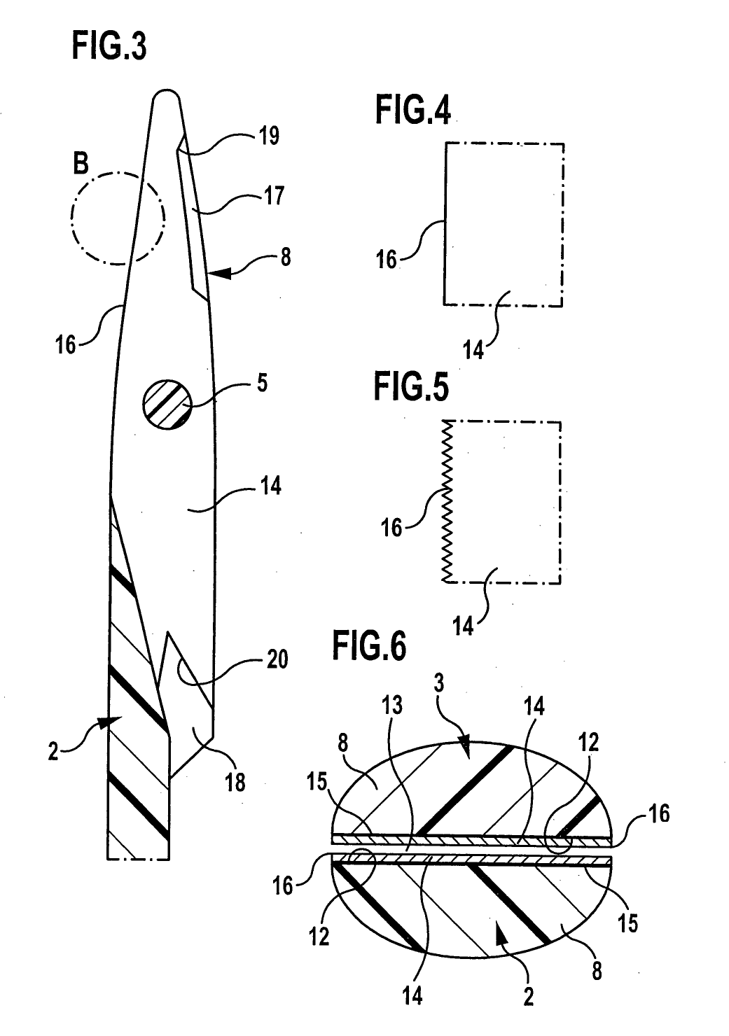

The scissors 1 shown in the drawings comprise two branches 2, 3 which are pivotably connected to one another at a pivot bearing 4. In the illustrated embodiment, this pivot bearing is formed by a bearing pin 5 penetrating a branch 2 and protruding from this branch 2 in the direction towards the other branch 3. In this area, the other branch 3 has a bearing opening 6 in which the bearing pin 5 engages. To axially fix the branch 3 on the bearing pin 5 there is arranged on the branch 2 a laterally protruding lug 7 which engages over the branch 3 when both branches 2, 3 extend parallel to one another in the closed position, as shown in unbroken lines in FIG. 1. When, however, the branch 3 is pivoted through approximately 90° in relation to this closed position (shown in dot-and-dash lines in FIG. 1) it then no longer dips in below the lug 7 and is readily removable from the bearing pin 5. Assembly of the two branches 2, 3 without a tool is thus possible, and in the normal working position the branches 2, 3 are fixed in axial direction relative to one another by the lug 7.

In the embodiment shown in the drawings, the bearing pin 5 is in the form of a separate component. It may, however, be integrally formed on the branch 2.

Each of the two branches 2, 3 has a cutting section 8 arranged at the front end of the branches 2, 3, a handle section 9 with finger loops 10 at the opposite end and a connecting section 11 between the cutting section 8 and the handle section 9.

In the cutting section 8, the two branches 2, 3 are each provided with an inner surface 12 pointing towards the respective other branch. This inner surface 12 is of slightly concave configuration, thereby producing a narrow slit 13 (FIG. 2) between the two inner surfaces 12 of the opposite branches 2, 3. The pivot bearing 4 is located approximately at the center of the longitudinal extent of these inner surfaces, and the cutting sections 8 of the two branches 2, 3 are pressed elastically against one another by the pivot bearing 4 and the lug 7, respectively, whereby the width of the slit 13 is reduced.

In the embodiment shown in the drawings, the two branches 2, 3 are each of integral construction and consist of a plastic material, for example, of polyamide (PA, PPA), of polyetheretherketone (PEEK) or of a liquid crystal polymer (LCP). This plastic material is preferably reinforced by embedding fibrous materials, for example, glass fibers or carbon fibers therein. With this construction, it is simple to integrally form the bearing pin 5 on branch 2 and the lug 7 on branch 3, so that as a whole the scissors are made up of only two parts.

A metal foil 14 is placed with surface-to-surface contact on each of the two inner surfaces 12. This metal foil 14 is adhesively bonded to the inner surface 12, for example, by a double-sided adhesive tape 15 (FIG. 6) placed therebetween, which may be coated with an adhesive which polymerizes under the action of heat.

The metal foil 14 consists of a metal with a high degree of hardness, in particular, with a hardness of at least HRC60. It is expedient to use a spring steel as metal. The thickness of the metal foil 14 is between 0.05 and 0.4 mm. The shape of the metal foil 14 corresponds to the shape of the inner surface 12. The metal foil 14 is in alignment with the side edge of the inner surface 12 or at the most protrudes very slightly over it, and the edges of the metal foils 14 which slide along one another upon opening the branches 2, 3 form the cutting edges 16 of the scissors 1.

The metal foils 14 are produced from a larger metal foil, for example, by punching-out, wire eroding or laser cutting, and can be placed without any further machining on the inner surfaces 12 and adhesively bonded thereto. To achieve a good positioning of the metal foils 14, the inner surfaces 12 have raised projections 17, 18, which project in the direction towards the inner surface of the opposite branch and engage in complementary recesses 19 and 20, respectively, of the metal foil 14. One projection 17 runs along the edge of the inner surface 12 opposite the cutting edge 16 and extends over part of the length of this edge. The other projection 18 is arranged at the handle section end of the inner surface 12 and is wedge-shaped in the direction towards the front end of the scissors 1, so that upon placing the metal foil 14 on the inner surface 12, an exact alignment of the metal foil 14 relative to the inner surface 12 is already brought about by the projections 17 and 18. The adhesive bonding can then be carried out in this position.

The cutting edge 16 formed by the metal foil 14 may be smooth (FIG. 4) or in a modified embodiment tooth-shaped (FIG. 5), so that the material which is to be cut is thereby held between the cutting edges 16 and secured against slipping.

It is expedient for the bonding of the metal foil 14 to the inner surface 12 to be carried out by means of an adhesive which is activatable under the action of heat. With branches made from a thermoplastic material, it is thus possible to perform the bonding during the manufacturing process of the branches or immediately thereafter, with the heat of formation of the branches being used to activate the adhesive.

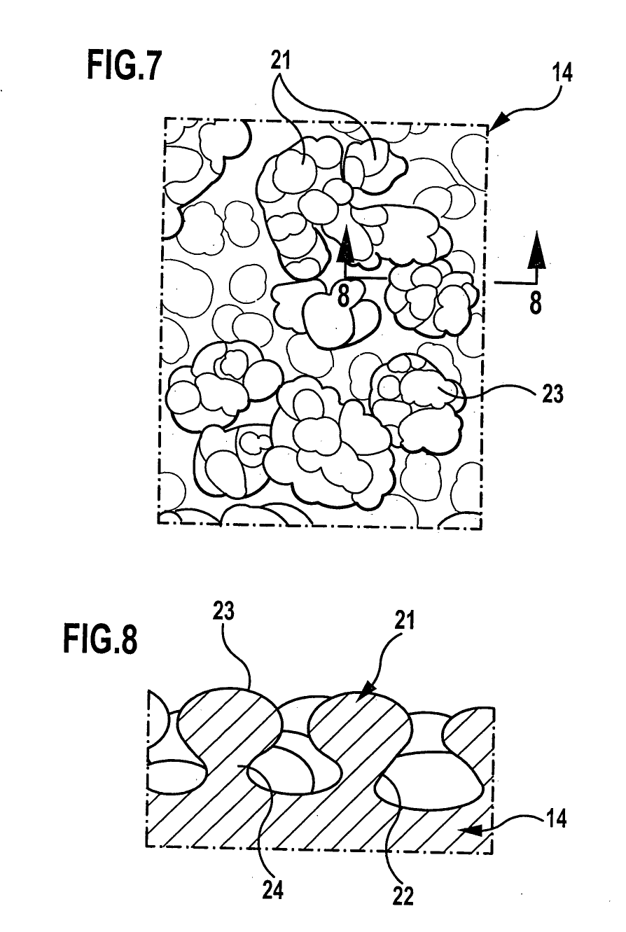

The connection between the metal foil and the inner surface of the branch can be further improved by the metal foil 14 carrying projections 21, preferably with undercuts 22, on its side facing the inner surface 12 of the branches 2, 3. As a rule, this will be a microstructure, which is shown on a highly enlarged scale in FIGS. 7 and 8. The projections may be distributed in a totally irregular manner over the surface of the metal foil 14. The structure may, for example, be of cauliflower-like configuration, i.e., the projections 21 have a spherical or generally convex outer surface 21 and are joined by a stem-like connection 24 to the metal foil 14. This surface structure may, for example, be produced by an etching process, or particularly advantageously by electrolytic deposition of an adhesive layer on the metal foil, in particular, an adhesive layer consisting of nickel.

To join it to the branch 2, 3, the metal foil 14 with its surface structured in this way is pressed flat against the inner surface 12 of the branch 2, 3, if required, with the additional application of heat, whereupon the plastic material of the branches 2, 3 flows and interlocks with the projections 21 and their undercuts 22. A flat and uniformly durable connection over the entire surface between the metal foil 14 and the inner surfaces 12 of the branches 2, 3 is thereby obtained after the cooling.

1. Scissors, in particular, for surgical purposes, comprising two branches pivotable relative to one another, each of which has a cutting section with a cutting edge at the front end thereof and a handle section at the rear end thereof, wherein at least one of the branches is joined in its cutting section on its inner surface facing the other branch to a metal foil lying flat thereon and extending at least at the cutting edge end of the inner surface as far as this cutting edge end or projecting slightly over this, and the edge of the metal foil arranged at this end of the inner surface forms the cutting edge of this inner surface, and wherein the metal foil has a thickness of between 0.05 and 0.4 mm.

2. Scissors in accordance with claim 1, wherein the metal foil consists of spring steel.

3. Scissors in accordance with claim 1, wherein the metal foil consists of a metal with a high degree of hardness.

4. Scissors in accordance with claim 1, wherein the metal foil is a punched part.

5. Scissors in accordance with claim 2, wherein the metal foil is a punched part.

6. Scissors in accordance with claim 1, wherein the metal foil is a laser-cut part.

7. Scissors in accordance with claim 1, wherein the metal foil is a wire-eroded part.

8. Scissors in accordance with claim 1, wherein the metal foil is adhesively bonded to the inner surface of the branch.

9. Scissors in accordance with claim 1, wherein a thermally polymerizable adhesive is arranged between metal foil and inner surface.

10. Scissors in accordance with claim 1, wherein a hot-melt adhesive is arranged between metal foil and inner surface.

11. Scissors in accordance with claim 1, wherein a double-sided adhesive surface is arranged between metal foil and inner surface.

12. Scissors in accordance with claim 1, wherein the metal foil is soldered or welded to the inner surface of the branch.

13. Scissors in accordance with claim 1, wherein the metal foil is joined to the inner surface of the branch by injecting the material of the branch around the metal foil.

14. Scissors in accordance with claim 2, wherein the metal foil is joined to the inner surface of the branch by injecting the material of the branch around the metal foil.

15. Scissors in accordance with claim 13, wherein the metal foil carries projections on the side thereof facing the inner surface of the branch, which dip into the material of the branch.

16. Scissors in accordance with claim 14, wherein the metal foil carries projections on the side thereof facing the inner surface of the branch, which dip into the material of the branch.

17. Scissors in accordance with claim 15, wherein the projections comprise undercuts.

18. Scissors in accordance with claim 15, wherein the projections are distributed in an irregular manner over the metal foil.

19. Scissors in accordance with claim 13, wherein the projections are formed by a layer of adhesive applied electrolytically to the metal foil.

20. Scissors in accordance with claim 13, wherein the projections consist of nickel.

21. Scissors in accordance with claim 13, wherein the projections are formed by the metal foil undergoing an etching treatment.

22. Scissors in accordance with claim 1, wherein the metal foil and the inner surface of the branch are joined together by hot-stamping.

23. Scissors in accordance with claim 1, wherein the edge forming the cutting edge of the metal foil comprises projections and recesses lying adjacent one another.

24. Scissors in accordance with claim 23, wherein the projections and recesses are tooth-shaped.

25. Scissors in accordance with claim 1, wherein the inner surface has projections raised in the direction towards the opposite branch, which engage in cutouts in the metal foil and thereby position this on the inner surface.

26. Scissors in accordance with claim 25, wherein a projection is arranged at the edge of the inner surface opposite the cutting edge.

27. Scissors in accordance with claim 26, wherein the projection extends parallel to the edge of the inner surface over part of the length of this edge.

28. Scissors in accordance with claim 25, wherein a projection is arranged at the handle section end of the inner surface.

29. Scissors in accordance with claim 28, wherein this projection converges in the direction towards the front end of the inner surface.

30. Scissors in accordance with claim 28, wherein the projection is of wedge-shaped design.

31. Scissors in accordance with claim 1, wherein the inner surfaces are of concave shape and the cutting sections of the two branches are resiliently pressed against one another.

32. Scissors in accordance with claim 1, wherein the cutting sections have a setting.

33. Scissors in accordance with claim 1, wherein the branches consist of a plastic material.

34. Scissors in accordance with claim 1, wherein the plastic material is reinforced with fibers.

35. Scissors in accordance with claim 1, wherein the inner surfaces of the branches extend with the metal foil in the direction towards their handle sections beyond the pivot connection of the branches.

36. Scissors in accordance with claim 35, wherein the pivot connection is arranged in the middle part of the inner surfaces provided with the metal foil.
